(19) United States
(12) Patent Application Publication
(10) Pub. No.: US 2020/0255811 A1
(43) Pub. Date: Aug. 13, 2020

(54) PHOSPHOTRIESTERASE ENZYME TETHERED MONOMER

(71) Applicant: The Government of the United States, as Represented by the Secretary of the Army, Fort Detrick, MD (US)

(72) Inventor: Stephen Drew KIRBY, Felton, PA (US)

(21) Appl. No.: 16/783,841

(22) Filed: Feb. 6, 2020

Related U.S. Application Data
(60) Provisional application No. 62/802,417, filed on Feb. 7, 2019.

(51) Int. Cl. C12N 9/16; C07K 14/245; C12N 15/86

(52) U.S. Cl. CPC C12N 9/16; C12Y 301/08001; A61K 38/00; C12N 15/86; C07K 2319/24; C07K 14/245

(57) ABSTRACT

Novel phosphotriesterase enzyme (PTE) monomers linked via an amino acid polypeptide are provided, expressed as a singular polypeptide with both subunits of the dimer attached from about 10 to 35 amino acids in length. This novel PTE enzyme has greater stability and/or enhanced activity in comparison to native forms of the enzyme. The novel PTE enzyme act as improved prophylactic medical counter measures against chemical nerve agents, as well as for use as decontaminants, bioscavengers for disposition in animal feedstocks, and components in assay kits.

### CROSS REFERENCE TO RELATED APPLICATIONS

This application claims the benefit of U.S. Provisional Application Ser. No. 62/802,417, filed Feb. 7, 2019, the contents of which are herein incorporated by reference in their entirety.

### STATEMENT OF GOVERNMENT SUPPORT

This invention was made by an employee of the U.S. Army Medical Research and Materiel Command, an agency of the U.S. government. Accordingly, the U.S. government has certain rights in the invention.

### REFERENCE TO SEQUENCE LISTING SUBMITTED ELECTRONICALLY

The official copy of the sequence listing is submitted electronically via EFS-Web as an ASCII formatted sequence listing with a file named “3000050-004000_Seq_Listing_ST25.txt”, created on Feb. 3, 2020 and having a size of 58,369 bytes and is filed concurrently with the specification. The sequence listing contained in this ASCII formatted document is part of the specification and is herein incorporated by reference in its entirety.

### FIELD OF THE INVENTION

The present invention relates to novel phosphotriesterase enzyme (such as organophosphate hydrolase (OPH)) monomers joined via an amino acid linker, expressed as a single polypeptide with both subunits of the dimer attached by a peptide linker from 10 to 35 amino acids in length, and a method of making same. In particular, the instant invention provides novel phosphotriesterase enzymes (PTEs) with greater stability and/or enhanced activity in comparison to native forms of the enzyme. The novel PTEs act as improved prophylactic medical counter measures against chemical nerve agents, as well as for use as decontaminants, bioscavengers for disposition in animal feedstocks, and components in assay kits.

### BACKGROUND OF THE INVENTION

Organophosphorous compounds, also known as organophosphates (OPs), are a class of compounds that comprise many commercial pesticides, as well as military-grade nerve gas agents. Organophosphates exhibit physiological toxicity by inactivating acetylcholinesterase (AChE) by binding to their active site, which leads to accumulation of acetylcholine and subsequent hyper-stimulation (and thus improper function) of nerve synapses. A fraction of an ounce (1 to 10 mL) of sarin—a nerve agent—on the skin can be fatal. Methods of dissemination include air, water, food, and agricultural contamination. Both inhalation and skin exposure to sarin produce health effects within 1 to 10 minutes. Current methods of neutralization of these chemicals on contaminated surfaces are resigned to the application of either detergents (with copious amounts of water) or caustic/industrial strength cleansers.

It has, however, been found that phosphotriesterase enzymes (PTEs) found in nature are capable of hydrolyzing OPs, including pesticides and nerve gas agents. However, these naturally occurring enzymes have insufficient enzymatic activity to neutralize OPs in a manner rapid and efficient enough to be effective for medical countermeasure use. Accordingly, it is an object of the present invention to provide a medical countermeasure against OPs, and in particular a PTE optimized for stability and efficacy which may be integrated into a deployment-ready solution.

### SUMMARY OF THE INVENTION

The present invention provides engineered PTEs as a monomer. PTEs, such as OPH, an enzyme from the bacteria Pseudomonas diminuta, typically exist as a homodimeric proteins. Two identical protein polypeptides come together through non-covalent interactions to form the holoenzyme. The present invention creates the same holoenzyme using only one polypeptide to encode the entire protein. In contrast to the naturally occurring enzyme, the two identical halves of the enzyme (i.e. the subunits) of the present invention are linked together using a flexible amino acid linker of a fixed length. In other embodiments, subunits from two different PTEs are linked together using a flexible amino acid linker of a fixed length to form a heterodimeric protein. The linker adds stability to the holoenzyme without detriment to activity and may enhance secretion from mammalian cells, allow for the development of asymmetric fusion partners, and allow for the development of PTE hybrids which otherwise inherently lack stability.

Accordingly, in a first embodiment of the present invention, a phosphotriesterase (PTE) dimer enzyme comprised of two phosphotriesterase subunits tethered to one another via an amino acid linker to form a tethered PTE monomer, wherein the amino acid linker is a polypeptide comprised of 10 to 35 amino acids.

In a second embodiment of the present invention, the phosphotriesterase dimer enzyme of the first embodiment above is provided, wherein the amino acid linker is a polyglycine linker.

In a third embodiment of the present invention, the amino acid linker of the first and second embodiment above is either from about 10 to about 35 amino acids in length.

In a fourth embodiment of the present invention, the amino acid linker of the first through third embodiment above is either 10 or 35 amino acids in length.

In some embodiments, the two phosphotriesterase subunits are identical, thus forming a homodimer.

In other embodiments, the two phosphotriesterase subunits are different, thus forming a heterodimer. For example, the two phosphotriesterase subunits may be two different mutant forms of PTE. Alternatively, the two phosphotriesterase subunits may be derived from two different phosphotriesterase enzymes, optionally from two different organisms.

Methods of preventing and/or treating organophosphate poisoning by administering the phosphotriesterase dimer enzymes of the invention are also provided.

Kits comprising the phosphotriesterase dimer enzymes of the invention are also provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain embodiments of the present invention. The invention may be better understood by reference to one or more of these drawings in combination with the detailed description of specific embodiments presented herein.

FIG. 1 shows a molecular model of organophosphate hydrolase (EC 3.1.8.1), a homodimeric, binuclear metalloenzyme. Subunit A is in blue and subunit B is in green; divalent metal ions are in red.

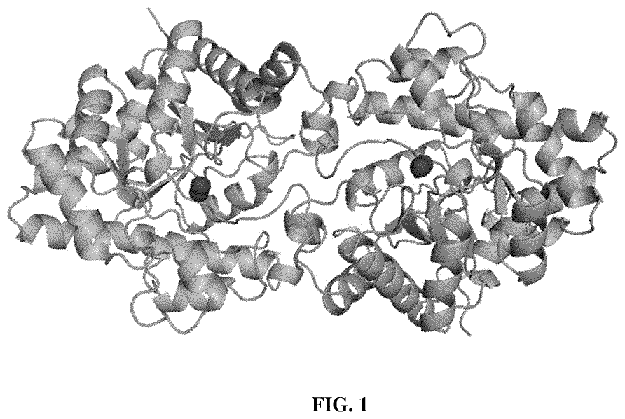

FIG. 2 is a graph showing growth kinetics of OPH T10 and OPH T35 examining enzymatic activity against paraoxon over the time of growth to determine optimal expression time needed for each construct.

FIG. 3 is a photo of a gel chromatograph showing PAGE for OPH T10 (A) and OPH T35 (B) for each step in the purification process: crude lysate (lane 2), cleared lysate (lane 3), flow through from resin (lane 4), wash 1 (lane 5), wash 2 (lane 6), elute pre-dialysis (lane 7), and elute post-dialysis (lane 8). Precision PLUS dual color protein standard in Lane 1 with the molecular weights listed in kDa.

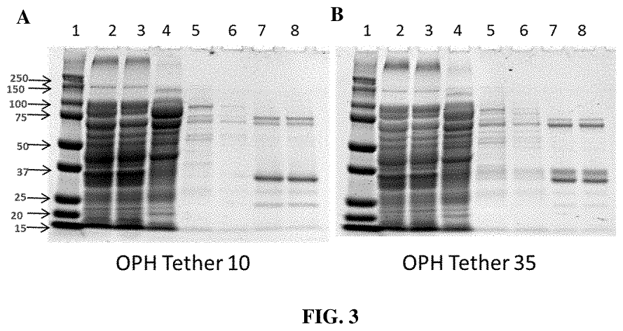

FIG. 4 is a photo of a gel chromatograph showing Coomassie blue protein gel (A) and Western blot (B) under denaturing conditions for OPH T10 (lane 2), OPH T35 (lane 3), and untethered OPH positive control (lane 4). Precision PLUS dual color protein standard in Lane 1 with the molecular weights listed in kDa.

FIG. 5 is a photo of a gel chromatograph showing Coomassie blue protein gel (A) and Western blot (B) under native conditions for OPH T10 (lane 2), OPH T35 (lane 3), and untethered OPH positive control (lane 4). Precision PLUS dual color protein standard in Lane 1 with the molecular weights listed in kDa.

FIG. 6 is a graph showing dynamic light scattering of OPH T35 (1.4 μg/μL; 0.02 μm filtered) shows two populations of particles in solution. The vast majority of protein (99.9% mass) can be found in the 8.6 nm population which has a calculated molecular weight of 514 kDa. This is consistent with the native Western blot for OPH T35 (FIG. 5, lane 3). Both particle populations show a % polydispersity (%Pd) greater than 20%, suggesting that neither population is monodispersed.

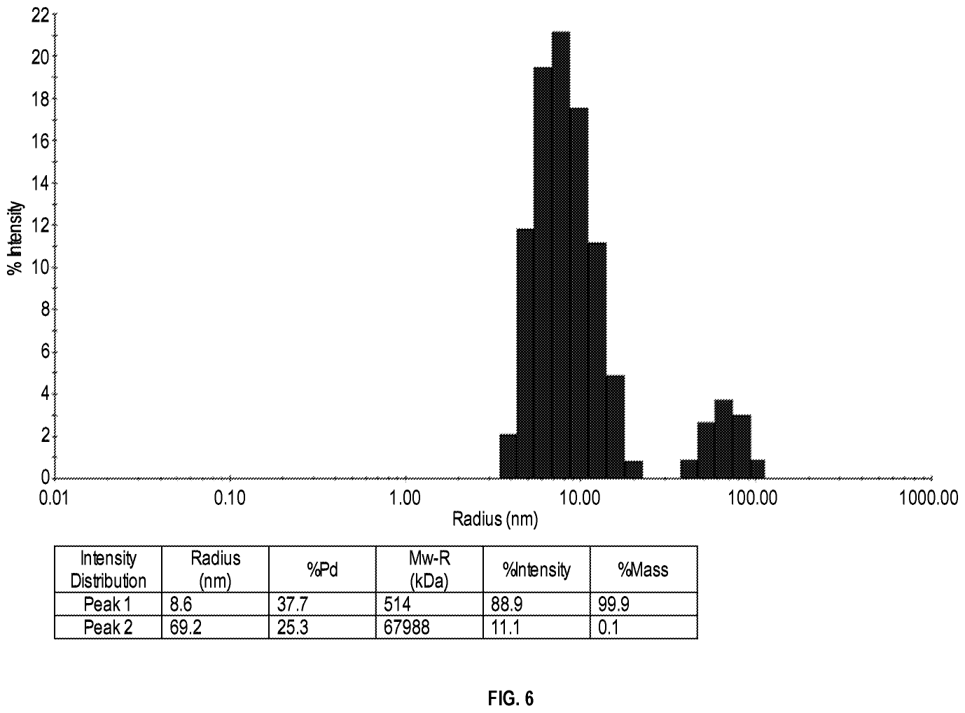

FIG. 7 is an illustration showing molecular space filled models of OPH T10 (A) and OPH T35 (B) & (C). OPH is indicated by grey spheres and the T10 or T35 tethers are indicated by colored spheres. Two alternative positions for the 35× glycine tether are provided in panels B and C. In panel B, the tether remains above the OPH dimer and may even twist back on itself as it rises above the protein mass. In panel C, the 35× glycine tether wraps around the protein mass and fits tight along a ridge of the protein partially blocking one of the two active sites (green sphere).

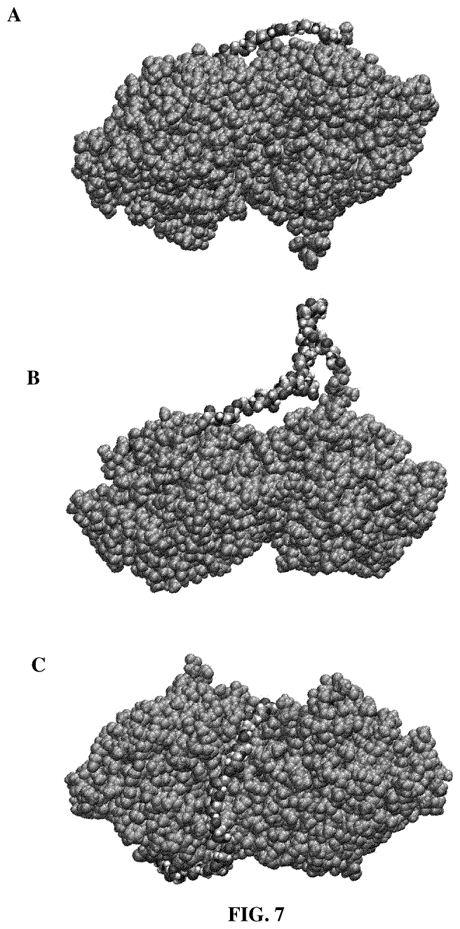

FIG. 8 is a graph showing size measurements for OPH T10 and OPH T35 compared to non-tethered OPH.

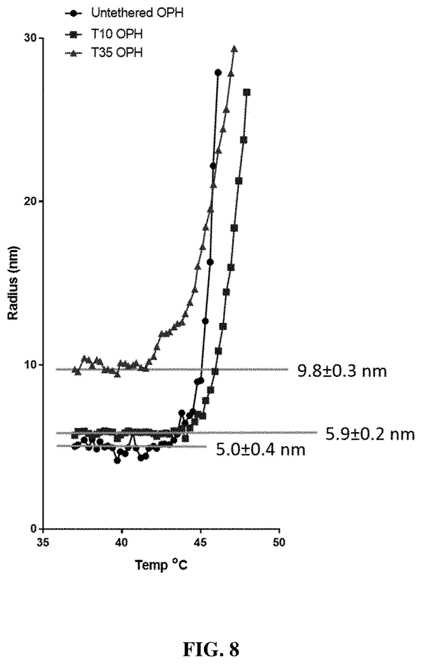

FIG. 9 is a graph showing results from a UPC2 assay against cyclosarin (O-methyl cyclohexyl phosphonofluoridate) for OPH T10 (B) and OPH T35 (C) compared to untethered OPH (YTRN) (A).

FIG. 10 is a graph showing results from a UPC2 assay against sarin (O-isopropylmethyl phosphonofluoridate) for OPH T10 (B) and OPH T35 (C) compared to untethered OPH (YTRN) (A).

FIG. 11 is a graph showing thermal melting results for OPH T10 and OPH T35 compared to untethered OPH (UN).

FIG. 12 is a graph showing results from a UPC2 assay against cyclosarin (O-methyl cyclohexyl phosphonofluoridate) for an OPH/IVH3 T35 heterodimer.

FIG. 13 is a graph showing size measurements for OPH/IVH3 T35 heterodimer compared to OPH T10 homodimer, OPH T35 homodimer, and non-tethered OPH homodimer.

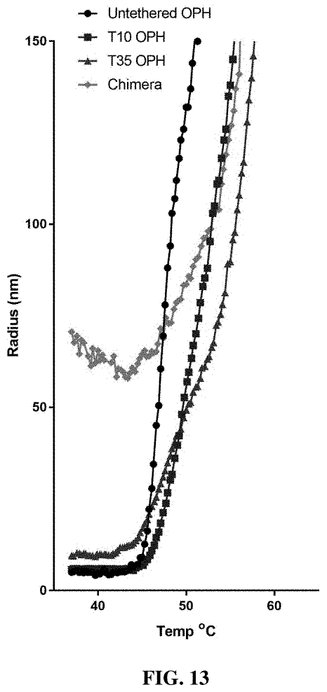

### DETAILED DESCRIPTION OF THE INVENTION

As mentioned above, phosphotriesterase (PTE) mutants have shown potential use as a medical countermeasure against organophosphorus compounds (OPs). PTE is typically expressed in bacteria as a homodimer; for example, an enzyme from the bacteria Pseudomonas diminuta (OPH), typically exists as a homodimeric protein. In particular, two separate OPH subunits (35 kDa each) self-assemble through non-covalent bonding at one a mirrored face of the enzyme, close to the putative active site, i.e., two identical protein polypeptides come together through non-covalent interactions to form the holoenzyme. However, PTE homodimers do not secrete expediently from mammalian cells. This causes potential problems when trying to express the protein from a heterologous plasmid or viral delivery system in mammalian cells. To enhance secretion, the present inventor sought to increase protein solubility without catastrophic detriment to activity and without additional fusion proteins.

In order to overcome these deficiencies in naturally occurring PTEs, the present inventor endeavored to develop a PTE operable to act as a nerve agent bioscavenger by protecting acetylcholinesterase from inhibition, and thus reducing lethality of nerve agents to those exposed thereto. As a result, the present inventor discovered an engineered PTE as a “unified” monomer.

This invention creates a holoenzyme using only one polypeptide to encode the entire holoenzyme. The two halves of the enzyme are linked together using a flexible amino acid linker of a fixed length of from between about 10 and 35 amino acids. The linker adds stability to the holoenzyme and for developing PTE hybrids which may otherwise inherently lack stability.

In particular, the PTE of the present invention is expressed as a monomer by joining two subunits with a poly-glycine linker. The result is a single polypeptide PTE with a tether 10 or 35 amino acids in length joining the two halves, and named them T10 and T35 respectively. Western blot analysis and paraoxon hydrolysis assays revealed that T10 was being produced and retained some activity against paraoxon. This was a surprise as we expected T10 to have no enzymatic activity. T35 monomer (75 kDa) was also being produced and retained 71% of specific activity against paraoxon compared to untethered OPH. T10 and T35 showed no significant decrement in activity against the nerve agent sarin and enhanced activity against cyclosarin. Both constructs showed high molecular weight aggregates greater than 250 kDa in dynamic light scattering and native polyacrylamide gels. These tethered constructs are the first attempts known for producing PTEs, such as OPH, as a single polypeptide.

In some aspects, the two subunits of the tethered monomer are identical (homodimer). In other aspects, the two subunits of the tethered monomer are different (heterodimer).

In some aspects, the two subunits are independently selected from any PTE or mutant thereof. As used herein, the term “phosphotriesterase”, also referred to as parathion hydrolase or organophosphorus hydrolase (EC: 3.1.8.1), refers to an enzyme belonging to the amidohydrolase superfamily. The bacterial enzyme phosphotriesterase (PTE) from Pseudomonas diminuta has been the subject of extensive interrogation due to its ability to hydrolyze a wide array of neurotoxic organophosphate compounds. While wild-type PTE has reasonable activity against the G-type nerve agents (kcat/Km-10⁵ M-1 s-1), this enzyme preferentially hydrolyzes the less toxic Rp-enantiomers. Directed evolution of PTE to specifically target the G-type nerve agents has led to the identification of the variant H257Y/L303T (YT), which has proven highly efficient at the hydrolysis of the more toxic S_{P}-enantiomer of sarin (GB), soman (GD), and cyclosarin (GF) with values of kcat/Km that exceed 10⁶ M-1 s-1. Many further variants of PTE have been developed to target G-type and/or V-type nerve agents and are known in the art. Any of these PTE mutants can be used as subunits in the tethered PTE monomers of the invention.

In some embodiments, each subunit is independently selected from a PTE comprising an amino acid sequence at least 80% identical, at least 85% identical, 90% identical, at least 91% identical, at least 92% identical, at least 93% identical, at least 94% identical, at least 95% identical, at least 96% identical, at least 97% identical, at least 98% identical, at least 99% identical to the sequence of the wild-type PTE from Pseudomonas diminuta as set forth in SEQ ID NO: 1 or a functional fragment thereof.

In some embodiments, each subunit is independently selected from a PTE comprising an amino acid sequence at least 80% identical, at least 85% identical, 90% identical, at least 91% identical, at least 92% identical, at least 93% identical, at least 94% identical, at least 95% identical, at least 96% identical, at least 97% identical, at least 98% identical, at least 99% identical to the sequence of a mutant PTE (YTRN) as set forth in SEQ ID NO: 2 or a functional fragment thereof.

In some embodiments, each subunit is independently selected from a PTE comprising an amino acid sequence at least 80% identical, at least 85% identical, 90% identical, at least 91% identical, at least 92% identical, at least 93% identical, at least 94% identical, at least 95% identical, at least 96% identical, at least 97% identical, at least 98% identical, at least 99% identical to the sequence of a mutant PTE (IVH3) as set forth in SEQ ID NO: 3 or a functional fragment thereof.

In the context of the present application, the “percentage of identity” or “percent identity” is calculated using a global pairwise alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the invention, is calculated using the EMBOSS: needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 91%, 92%, 93%, 94%, 95%, 96%, 97%, 98% or 99% identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

“Amino acid substitutions” may be conservative or non-conservative. Preferably, substitutions are conservative substitutions, in which one amino acid is substituted for another amino acid with similar structural and/or chemical properties.

In an embodiment, conservative substitutions may include those, which are described by Dayhoff in “The Atlas of Protein Sequence and Structure. Vol. 5”, Natl. Biomedical Research, the contents of which are incorporated by reference in their entirety. For example, in an aspect, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In an aspect, a conservative amino acid substitution may be selected from the following of T→A, G→A, A→I, T→V, A→M, T→I, A→V, T→G, and/or T→S.

In a further embodiment, a conservative amino acid substitution may include the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln.

In another embodiment, conservative substitutions may be made in accordance with Table A. Methods for predicting tolerance to protein modification may be found in, for example, Guo et al., Proc. Natl. Acad. Sci., USA, 101 (25):9205-9210 (2004), the contents of which are incorporated by reference in their entirety.

TABLE A
Conservative Amino Acid substitution
Conservative Amino Acid Substitutions

| Amino Acid | Substitutions (others are known in the art) |
|---|---|
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

In an aspect, sequences described herein may include 1, 2, 3, 4, 5, 10, 15, 20, 25, or 30 amino acid or nucleotide mutations, substitutions, deletions. In yet another aspect, the mutations or substitutions are conservative amino acid substitutions.

In an aspect, both subunits of the tethered PTE monomer comprise the amino acid sequence of SEQ ID NO: 1 or a variant and/or functional fragment thereof as defined herein.

In an aspect, both subunits of the tethered PTE monomer comprise the amino acid sequence of SEQ ID NO: 2 or a variant and/or functional fragment thereof as defined herein.

In another aspect, both subunits of the tethered PTE monomer comprise the amino acid sequence of SEQ ID NO: 3 or a variant and/or functional fragment thereof as defined herein.

In yet another aspect, one subunit of the tethered PTE monomer comprises the amino acid sequence of SEQ ID NO: 1 or a variant and/or functional fragment thereof as defined herein and the other subunit of the tethered PTE monomer comprises an amino acid sequence selected from the group consisting of SEQ ID NO: 2, SEQ ID NO: 3, and a variant and/or functional fragment thereof as defined herein.

In yet another aspect, one subunit of the tethered PTE monomer comprises the amino acid sequence of SEQ ID NO: 2 or a variant and/or functional fragment thereof as defined herein and the other subunit of the tethered PTE monomer comprises an amino acid sequence selected from the group consisting of SEQ ID NO: 1, SEQ ID NO: 3, and a variant and/or functional fragment thereof as defined herein.

In yet another aspect, one subunit of the tethered PTE monomer comprises the amino acid sequence of SEQ ID NO: 3 or a variant and/or functional fragment thereof as defined herein and the other subunit of the tethered PTE monomer comprises an amino acid sequence selected from the group consisting of SEQ ID NO: 1, SEQ ID NO: 2, and a variant and/or functional fragment thereof as defined herein.

In a preferred embodiment, both subunits of the tethered PTE monomer comprise the amino acid sequence of SEQ ID NO: 2.

Any suitable polypeptide can be used as a linker to join the two PTE subunits into a dimer. It will be understood by those of skill in the art that the size and/or sequence of the polypeptide linker can be modified to optimize the three-dimensional structure of the tethered PTE monomer. Preferably, a polypeptide linker is chosen such that it allows the two PTE subunits to dimerize and does not interfere with the active site of the PTE enzyme.

In one embodiment, the polypeptide linker is a polyglycine linker.

In some aspects, the polypeptide linker is from about 10 amino acids to about 35 amino acids in length. In some aspects, the polypeptide linker is from 10 amino acids to 35 amino acids in length.

In some aspects, the polypeptide linker is about 10 amino acids in length. In some aspects, the polypeptide linker is about 35 amino acids in length.

It will be apparent that the subunits may be linked in any order. For example, if the two subunits are different, the first subunit may be 5′ or 3′ to the polypeptide linker.

In some embodiments, the tethered PTE monomer comprises the amino acid sequence of SEQ ID NO: 5 (YTRN-T10-YTRN) or a variant and/or functional fragment thereof as described herein.

In some embodiments, the tethered PTE monomer comprises the amino acid sequence of SEQ ID NO: 6 (YTRN-T35-YTRN) or a variant and/or functional fragment thereof as described herein.

In some embodiments, the tethered PTE monomer comprises the amino acid sequence of SEQ ID NO: 7 (IVH3-T10-IVH3) or a variant and/or functional fragment thereof as described herein.

In some embodiments, the tethered PTE monomer comprises the amino acid sequence of SEQ ID NO: 8 (IVH3-T35-IVH3) or a variant and/or functional fragment thereof as described herein.

In some embodiments, the tethered PTE monomer comprises the amino acid sequence of SEQ ID NO: 9 (IVH3-T35-YTRN) or a variant and/or functional fragment thereof as described herein.

It will be appreciated that in order to aid in isolation of the protein, the protein may be expressed with one or more additional amino acid sequences (i.e., tags) engineered to enhance stability, production, purification, yield or toxicity of the expressed polypeptide. Such a fusion protein can be designed so that the fusion protein can be readily isolated by affinity chromatography; e.g., by immobilization on a column specific for the heterologous protein.

Examples of affinity tags include, but are not limited to, HIS, CBP, CYD (covalent yet dissociable NorpD peptide), Strep II, FLAG, HPC (heavy chain of protein C) peptide tags, and the GST and MBP protein fusion tag systems.

In some embodiments, at least one affinity tag is a HIS tag, for example a 6×HIS tag.

In some embodiments, at least one affinity tag is maltose binding protein (MBP). According to a particular embodiment, the affinity tag is an MBP comprising the amino acid sequence of SEQ ID NO: 4.

In some embodiments, more than one affinity tag is present.

The polypeptides of the present invention are preferably expressible in bacteria such as E. coli [e.g., BL21, BL21 (DE3), Origami B (DE3), available from Novagen (www(dot)calbiochem(dot)com) and RIL (DE3) available from Stratagene, (www(dot)stratagene(dot)com). Preferably, at least 2%, at least 5%, at least 10%, at least 20%, at least 30%, at least 40%, at least 50%, at least 60%, at least 70%, at least 80%, at least 90%, at least 95% or more, say 100%, of bacterially expressed protein remains soluble (i.e., does not precipitate into inclusion bodies).

The present invention also provides nucleic acid sequences encoding the tethered PTE polypeptides.

Thus, according to an aspect of the present invention there is provided an isolated polynucleotide comprising a nucleic acid sequence which encodes the tethered PTE polypeptides of the present invention.

Recombinant techniques are preferably used to generate the polypeptides of the present invention. Such recombinant techniques are well known in the art and are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

To produce a polypeptide of the present invention using recombinant technology, a polynucleotide encoding a polypeptide of the present invention is ligated into a nucleic acid expression construct, which includes the polynucleotide sequence under the transcriptional control of a cis-regulatory (e.g., promoter) sequence suitable for directing constitutive or inducible transcription in the host cells, as further described below.

Exemplary polynucleotide sequences for expressing the polypeptides of the present invention are set forth in SEQ ID NOs: 10-14.

Other than containing the necessary elements for the transcription and translation of the inserted coding sequence, the expression construct of the present invention can also include sequences (i.e., tags) engineered to enhance stability, production, purification, yield or toxicity of the expressed polypeptide. Such a fusion protein can be designed so that the fusion protein can be readily isolated by affinity chromatography; e.g., by immobilization on a column specific for the heterologous protein. Where a cleavage site is engineered between the peptide moiety and the heterologous protein, the peptide can be released from the chromatographic column by treatment with an appropriate enzyme or agent that disrupts the cleavage site [e.g., see Booth et al. (1988) Immunol. Lett. 19: 65-70; and Gardella et al., (1990) J. Biol. Chem. 265:15854-15859].

A variety of prokaryotic or eukaryotic cells can be used as host-expression systems to express the polypeptide coding sequence. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence. Mammalian expression systems can also be used to express the polypeptides of the present invention. Bacterial systems are preferably used to produce recombinant polypeptides, according to the present invention, thereby enabling a high production volume at low cost.

Other expression systems such as insects and mammalian host cell systems, which are well known in the art can also be used by the present invention.

In any case, transformed cells are cultured under effective conditions, which allow for the expression of high amounts of recombinant polypeptides. Effective culture conditions include, but are not limited to, effective media, bioreactor, temperature, pH and oxygen conditions that permit protein production. An effective medium refers to any medium in which a cell is cultured to produce the recombinant polypeptides of the present invention. Such a medium typically includes an aqueous solution having assimilable carbon, nitrogen and phosphate sources, and appropriate salts, minerals, metals and other nutrients, such as vitamins.

Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

Following a certain time in culture, recovery of the recombinant protein is effected. The phrase “recovering the recombinant protein” refers to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Polypeptides of the present invention can be used for treating an organophosphate exposure associated damage.

Thus, according to an aspect of the invention, there is provided a method of treating or preventing organophosphate exposure associated damage in a subject in need thereof, the method comprising providing the subject with a therapeutically effective amount of the tethered PTE polypeptide described above to thereby treat the organophosphate exposure associated damage in the subject.

As used herein, the term “treating” refers to preventing, curing, reversing, attenuating, alleviating, minimizing, suppressing or halting the deleterious effects of the immediate life-threatening effects of organophosphate intoxication and its long-term debilitating consequences.

As used herein, the phrase “organophosphate exposure associated damage” refers to short term (e.g., minutes to several hours post-exposure) and long term damage (e.g., one week up to several years post-exposure) to physiological function (e.g., motor and cognitive functions). Organophosphate exposure associated damage may be manifested by the following clinical symptoms including, but not limited to, headache, diffuse muscle cramping, weakness, excessive secretions, nausea, vomiting and diarrhea. The condition may progress to seizure, coma, paralysis, respiratory failure, delayed neuropathy, muscle weakness, tremor, convulsions, permanent brain dismorphology, social/behavioral deficits and general cholinergic crisis (which may be manifested for instance by exacerbated inflammation and low blood count. Extreme cases may lead to death of the poisoned subjects.

As used herein, the term “organophosphate compound” refers to a G-type and/or a V-type organophosphate (OP), as described herein.

As used herein, the phrase “a subject in need thereof” refers to a human or animal subject who is sensitive to OP toxic effects. Thus, the subject may be exposed or at a risk of exposure to OP. Examples include civilians contaminated by a terrorist attack at a public event, accidental spills in industry and during transportation, field workers subjected to pesticide/insecticide OP poisoning, truckers who transport pesticides, pesticide manufacturers, dog groomers who are overexposed to flea dip, pest control workers and various domestic and custodial workers who use these compounds, military personnel exposed to nerve gases.

As mentioned, in some embodiments of the invention, the method is effected by providing the subject with a therapeutically effective amount of the tethered PTE polypeptide (i.e., monomer) of the invention.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into animal cells are known in the art and including as non limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or viral vectors, in view of being expressed in cells.

By “delivery vector” or “delivery vectors” is intended any delivery vector which can be used in the present invention to put into cell contact (i.e, “contacting”) or deliver inside cells or subcellular compartments (i.e, “introducing”) molecules (proteins or nucleic acids) of the present invention. It includes, but is not limited to, liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection.

The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

By “lentiviral vector” is meant HIV-based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as non limiting example, that are able to integrate the genome of a target cell. At the opposite by “non integrative lentiviral vectors (or NILV)” is meant efficient gene delivery vectors that do not integrate the genome of a target cell through the action of the virus integrase.

Delivery vectors and vectors can be associated or combined with any cellular permeabilization techniques such as sonoporation or electroporation or derivatives of these techniques.

In some embodiments, the method comprises administering to the subject a vector comprising a polynucleotide sequence encoding the tethered PTE polypeptide of the invention.

In other embodiments, since OP can be rapidly absorbed from lungs, skin, gastro-intestinal (GI) tract and mucous membranes, the tethered PTE polypeptide of the invention may be provided by various administration routes or direct application on the skin.

For example, the tethered PTE polypeptide may be immobilized on a solid support e.g., a porous support which may be a flexible sponge-like substance or like material, wherein the PTE is secured by immobilization. The support may be formed into various shapes, sizes and densities, depending on need and the shape of the mold. For example, the porous support may be formed into a typical household sponge, wipe or tissue paper.

For example, such articles may be used to clean and decontaminate wounds, while the immobilized tethered PTE polypeptide will not leach into a wound. Therefore, the sponges can be used to decontaminate civilians contaminated by a terrorist attack at a public event.

Alternatively, or additionally, the tethered PTE polypeptide may be administered to the subject per se or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the tethered PTE monomer accountable for the biological effect.

As used herein, the phrases “physiologically acceptable carrier” and “pharmaceutically acceptable carrier,” which may be interchangeably used, refer to a carrier or a diluent that does not cause significant irritation to an organism and does not abrogate the biological activity and properties of the administered compound. An adjuvant is included under these phrases.

As used herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, dermal, transmucosal, especially transnasal, intestinal or parenteral delivery, including intramuscular, subcutaneous and intramedullary injections as well as intrathecal, direct intraventricular, intravenous, intraperiotoneal, intranasal, intrabone or intraocular injections.

Alternately, one may administer the pharmaceutical composition in a local rather than systemic manner, for example, via injection of the pharmaceutical composition directly into a tissue region (e.g., skin) of a patient. Topical administration is also contemplated according to the present teachings.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropyl methyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions. Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The tethered PTE monomers of the invention may be administered prior to the OP exposure (prophylactically, e.g., 10 or 8 hours before exposure), and alternatively or additionally administered post exposure, even days after (e.g., 7 days) in a single or multiple-doses.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The ability of PTE to sequester OP molecules, suggests use of the tethered PTE monomers of the invention in the decontamination of OP contaminated surfaces and detoxification of airborne OP.

Thus, an aspect of the invention further provides for a method of detoxifying a surface contaminated with an OP molecule; or preventing contamination of the surface with OP. The method is effected by contacting the surface with the tethered PTE monomers of the invention.

Thus, synthetic and biological surfaces contemplated according to embodiments of the invention include, but are not limited to, equipment, laboratory hardware, devices, fabrics (clothes), skin (as described above) and delicate membranes (e.g., biological). The mode of application will depend on the target surface. Thus, for example, the surface may be coated with foam especially when the surface comprises cracks, crevices, porous or uneven surfaces. Application of small quantities may be done with a spray-bottle equipped with an appropriate nozzle. If a large area is contaminated, an apparatus that dispenses a large quantity of foam may be utilized.

Coatings, linings, paints, adhesives sealants, waxes, sponges, wipes, fabrics which may comprise the tethered PTE monomers of the invention may be applied to the surface (e.g., in case of a skin surface for topical administration). Exemplary embodiments for such are provided in U.S. Patent Publication No. 20040109853.

Surface decontamination may be further assisted by contacting the surface with a caustic agent; a decontaminating foam, a combination of baking condition heat and carbon dioxide, or a combination thereof. Sensitive surfaces and equipments may require non corrosive decontaminants such as neutral aqueous solutions with active ingredient (e.g., paraoxonases).

In addition to the above described coating compositions, OP contamination may be prevented or detoxified using an article of manufacture which comprise the tethered PTE monomers immobilized to a solid support in the form of a sponge (as described above), a wipe, a fabric and a filter (for the decontamination of airborne particles). Chemistries for immobilization are provided in U.S. Patent Publication No. 20040005681, which is hereby incorporated in its entirety.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range.

All publications and patents mentioned herein are hereby incorporated by reference in their entirety as if each individual publication or patent was specifically and individually indicated to be incorporated by reference.

Various embodiments and aspects of the present invention as delineated hereinabove and as claimed in the claims section below find experimental support in the following examples.

### EXAMPLES

### Example 1

### OPH Homodimer Constructs

The main goal in combating organophosphorus (OP) poisoning is to protect acetylcholinesterase from inhibition, in both the peripheral and central nervous systems. This paradigm has been the cornerstone for protection against intoxication by OP nerve agents since their inception. Enzymes engineered to bind and catalytically degrade OP nerve agents have shown the most promise as alternative therapies in depleting nerve agent concentrations in the blood before they reach critical targets (Lenz et al., 2005). Typically mammalian enzymes in the blood have been chosen as platforms for developing catalytic bioscavengers; however, some variants of bacterially derived enzymes with catalytic efficiencies close to the diffusion limit offer the catalytic power needed to reduce toxic levels in a timely manner.

Organophosphate hydrolase (OPH; EC 3.1.8.1) has the highest catalytic capacity of any enzyme tested for hydrolyzing the OP paraoxon. Wild-type OPH (˜37 kDa; 325 amino acids) was initially purified from the bacteria Brevundimonas diminuta as a dimeric, binuclear metalloenzyme. One method for expression and delivery of this protein drug could be the use of adeno-associated viral vectors encoding an OPH sequence for production in organ tissues in vivo. This method can only be effective if the protein drug can be secreted into the blood and surrounding tissues. Enzymes, like paraoxonase 1 (PON1), under adenoviral control are readily expressed and secreted in an active form from tissues in vivo. Secretion has been difficult for OPH expression in mammalian cells in culture for unknown reasons.

We have attempted to promote OPH secretion from mammalian cells in culture by improving the solubility of the native enzyme. Kapust and Waugh (1999) showed that maltose binding protein (MBP), glutathione sulfurtransferase (GST), and thioredoxin (TXN) as fusion partners increased solubility. We produced these as fusion partners to OPH. Secreted GST-OPH was observed to be between 2.1% to 2.8% of the total activity produced in the cell. We observed maximal secretion using TXN-OPH and observed secretion to be 7.1% of total activity produced in cells.

To build upon the success achieved in expressing and secreting OPH with a fusion partner from mammalian cells, we propose to develop OPH as a functional monomer from a single polypeptide, thereby eliminating the engineered fusion protein. OPH is typically expressed in bacteria as a homodimer. Two monomers self-assemble through non-covalent bonding at the face close to the putative active site. We decided to express OPH as a single polypeptide with both subunits of the dimer attached by a flexible tether. Cheng et al. (1990) tethered the dimer of a human immunodeficiency virus protease and showed enhanced stability with no loss in activity. In cases where heterologous protein expression has been difficult, tethering proteins together has improved stability and solubility of the complex (Gadd et al., 2011; Fremont et al., 1996).

This approach to designing an OPH molecule as a single polypeptide has not been attempted until now. This OPH hybrid approach will provide us with the benefits of manipulating each subunit of OPH with mutations by which we can direct hydrolysis against one set of nerve agents while the other subunit can be engineered to preferentially hydrolyze another set. All of this can be achieved within one polypeptide, thereby reducing components for a drug for FDA IND approval.

### Materials and Methods

In vitro expression of OPH variants in E. coli:

OPH Tether 10 (SEQ ID NO: 10) and OPH Tether 35 (SEQ ID NO: 11) were tagged with a 6×HIS tag at the 3′ end and were synthesized by GenScript (Piscataway, N.J.) in a pET-20b(+) plasmid.

Escherichia coli BL21 (DE3) competent cells were transformed and grown as 1 L Terrific Broth cultures supplemented with 100 μg/mL ampicillin and 100 μM CoCl2; grown at 30° C. for ˜23 hours.

### OPH Purification Using Ni-NTA Resin

Cell pellets were re-suspended in a 5:1 ratio of lysis buffer [100 mM Tris pH 8.0, 10 mM NaHCO₃, 100 μM CoCl₂, 10% glycerol, 10 μL/mL Halt Protease Inhibitor, 400 μg/ml Lysozyme, and Benzonase], mixed at 4° C. for 1 hour, and disrupted by sonication; lysate was clarified by centrifugation and mixed with Ni-NTA overnight.

Wash buffer: 100 mM Tris pH 8.0, 100 mM NaCl, 25 μM CoCl₂, 10% glycerol, and 40 mM imidazole 3×.

Elute buffer: 100 mM Tris pH 8.0, 100 mM NaCl, 25 μM CoCl₂, 10% glycerol, and 175 mM imidazole.

Nano-drop A₂₈₀ for protein concentration and all fractions were assayed for paraoxonase activity; fractions containing significant paraoxonase activity were combined.

Buffer was exchanged using a 30 MWCO Dialysis Cassette (ThermoFisher) to 100 mM Tris pH 8.0, 100 mM NaCl, 25 μM CoCl₂, and 10% glycerol.

### OPH Activity Assays Using O,O,Diethyl p-Nitrophenol (Paraoxon)

The rate of formation of p-nitrophenol was monitored at A412 (ϵ=17,000 M⁻¹ cm⁻¹) for 5 minutes at 25° C. with 10 mM paraoxon in a 96-well plate using a SpectraMax (Molecular Devices) M5 series spectrophotometer.

### Gel Electrophoresis and Western Blot

10% PAGE for both denaturing and native conditions; Coomassie blue stain and Western blot analysis.

Gels were transferred to a nitrocellulose membrane using iBlot2 transfer system; membrane was blocked in Licor Odyssey blocking solution gently rocking for 1 hour at 4° C.; membrane was incubated with primary antibody (1:50,000 rabbit polyclonal antibody OPH whole molecule, unpurified; Washington Biotech, Baltimore, Md.) diluted in blocking solution overnight at 4° C.; membrane was rinsed 3 times for 5 minutes each with phosphate buffered saline (PBS) and PBS plus tween 20 (PBST); membrane was incubated with secondary antibody (1:5,000 goat anti-rabbit IRDye 680LT diluted in blocking solution) for 2 hours at 4° C.; membrane was rinsed 3 times for 5 minutes each with PBST, and washed a last time with PBS before imaging on the Licor Odyssey.

### Results

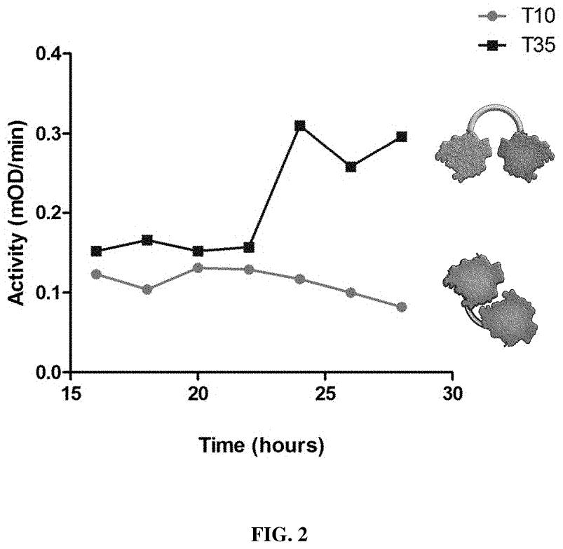

We produced in bacteria a single polypeptide encoding both subunits of the homodimeric protein known as organophosphate hydrolase (OPH or also known as phosphotriesterase or PTE) with a polyglycine linker 10 or 35 amino acids in length between the two subunits (FIGS. 2 & 3).

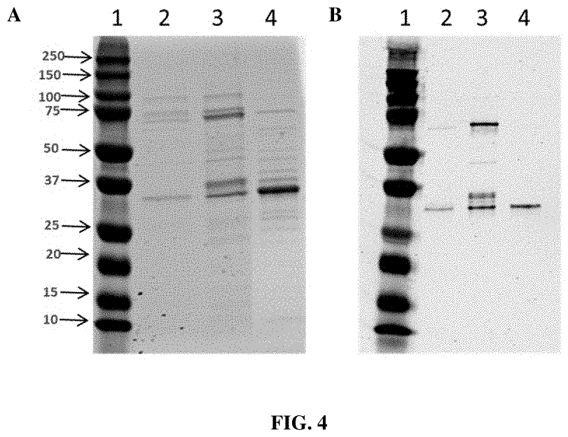

We observed a ˜73 kDa protein in denaturing gels for both T10 and T35 OPH, but also observed ˜35 kDa fragments, suggesting either breakage of the full-length protein at the linker or incomplete translation of the entire mRNA (FIG. 4).

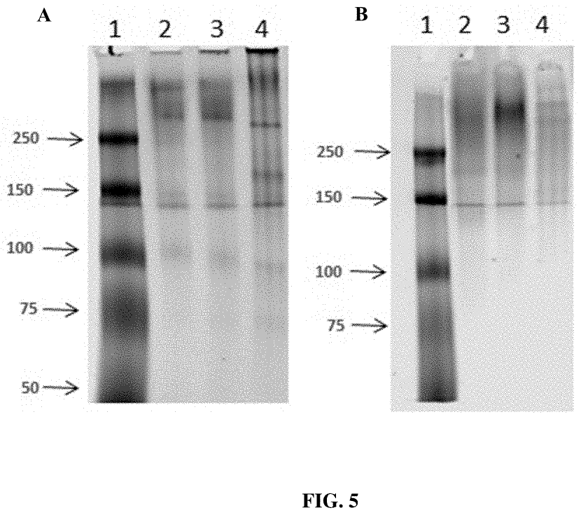

In native gels, OPH T10 and OPH T35 showed aggregates greater than 250 kDa, suggesting multimerization of the protein (FIG. 5).

The data from paraoxon hydrolysis activity assays for OPH T10 and OPH T35, an untethered OPH (positive control), and PBS (negative control) are presented below in Table 1. Each run is an average of three separate assays (n =3) and the 5th column shows the average specific activity in μ moles/min/mg protein±standard error of the mean (SEM). The last column shows percent activity of each construct compared to the untethered OPH activity.

TABLE 1

| | | | | Average Specific | % |
|---|---|---|---|---|---|
| | | | | Activity (μmoles/ | Untethered |
| Sample | Run A | Run B | Run C | min/mg) ± SEM | activity |
| negative | 0.01 | 0.01 | 0.01 | 0.01 ± 0.0 | 0 |
| control | | | | | |
| untethered | 852.71 | 725.21 | 744.64 | 774.19 ± 39.66 | 100 |
| OPH | | | | | |
| OPH T10 | 261.94 | 218.88 | 195.76 | 225.53 ± 19.39 | 29 |
| OPH T35 | 603.11 | 521.96 | 523.72 | 549.60 ± 26.76 | 71 |

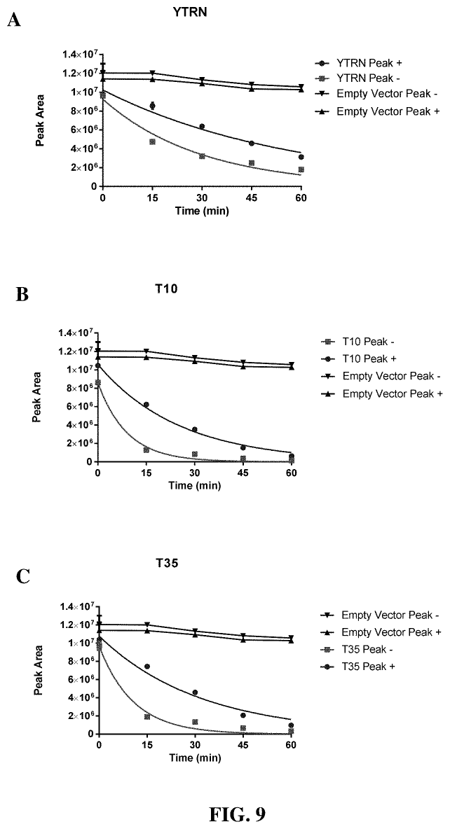

OPH T10 and T35 were also tested in a UPC2 assay against sarin (O-isopropylmethyl phosphonofluoridate) and cyclosarin (O-methyl cyclohexyl phosphonofluoridate), and compared to untethered OPH (YTRN) and empty vector control. The experiment was completed by SPC Jaffet Santiago Garcia and Mrs. Cetara Baker on May 21, 2018. Data was compiled and added to notebook 042-02, page 88, Protocol 1-01-02-000-A-814. This experiment demonstrated that both tethered OPH constructs (i.e. T10 and T35) showed substantial improvements (2× to 3× relative change) in catalytic efficiencies against the organophosphorus nerve agent cyclosarin with a slight improvement in stereoselectivity for the more toxic stereoisomer (e.g. P-) of cyclosarin (Table 2; FIG. 9A-C).

TABLE 2

| | Apparent Catalytic | | |
|---|---|---|---|
| Enzyme/ | Efficiency | Relative | Stereoselectivity |
| (GF isomer) | (M⁻¹ min⁻¹) | Change | (S_{P}/R_{P}) |
| YTRN (R_{P}) | 3.02E+05 | — | 1.93 |
| YTRN (S_{P}) | 5.82E+05 | — | |
| T10 (R_{P}) | 7.00E+05 | 2.3 | 2.82 |
| T10 (S_{P}) | 1.97E+06 | 3.4 | |
| T35 (R_{P}) | 5.48E+05 | 1.8 | 2.95 |
| T35 (S_{P}) | 1.62E+06 | 2.8 | |

* S_{P} denotes P(−) stereoisomer; R_{P} denotes P(+) stereoisomer

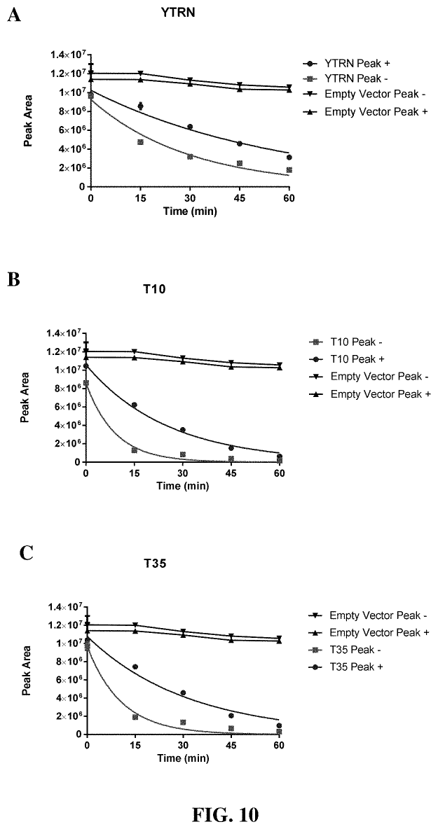

There is no significant detriment to activities against the nerve agent sarin away from the same mutant OPH untethered control (Table 3; FIG. 10A-C).

TABLE 3

| | Apparent Catalytic | | |
|---|---|---|---|
| Enzyme/ | Efficiency | Relative | Stereoselectivity |
| (GB isomer) | (M⁻¹ min⁻¹) | Change | (S_{P}/R_{P}) |
| YTRN (R_{P}) | 4.90E+05 | — | 1.45 |
| YTRN (S_{P}) | 7.09E+05 | — | |
| T10 (R_{P}) | 6.44E+05 | 1.3 | 1.11 |
| T10 (S_{P}) | 7.12E+05 | 1.0 | |
| T35 (R_{P}) | 6.52E+05 | 1.3 | 1.01 |
| T35 (S_{P}) | 6.59E+05 | 0.9 | |

* S_{P} denotes P(−) stereoisomer; R_{P} denotes P(+) stereoisomer

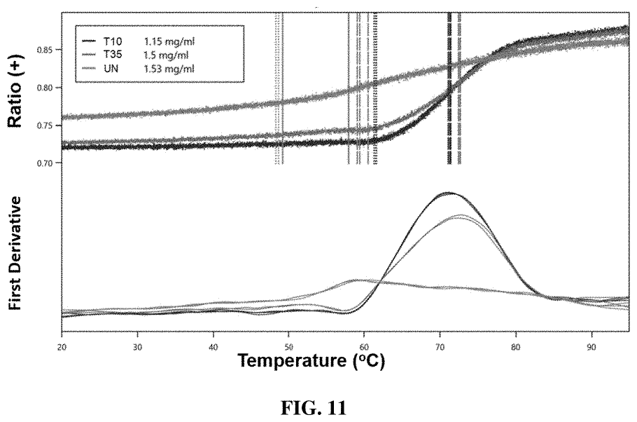

Thermal melting was measured for OPH T10 and T35 and compared to untethered OPH as shown in Table 4 and FIG. 11. Tethered OPH (T10 and T35) improves thermostability over untethered OPH by raising the TM ˜10° C.

TABLE 4

| | Untethered | T10 | T35 |
|---|---|---|---|
| Tₒₙₛₑₜ Unfolding (° C.) | 48.7 ± 0.4 | 61.6 ± 0.1 | 57.6 ± 0.5 |
| T_{M} (° C.) | 59.7 ± 0.6 | 71.3 ± 0.2 | 72.5 ± 0.1 |

### Example 2

### OPH/IVH3 Heterodimer Constructs

The same methods as described in Example 1 were used to construct and test a heterodimer containing one OPH subunit and one IVH3 subunit linked with a 35 amino acid polyglycine linker (SEQ ID NOS: 9 (aa) and 14 (nt)). IVH3 is a mutant OPH sequence designed to hydrolyze V-class organophosphorus nerve agents (i.e. VX, VR, etc.).

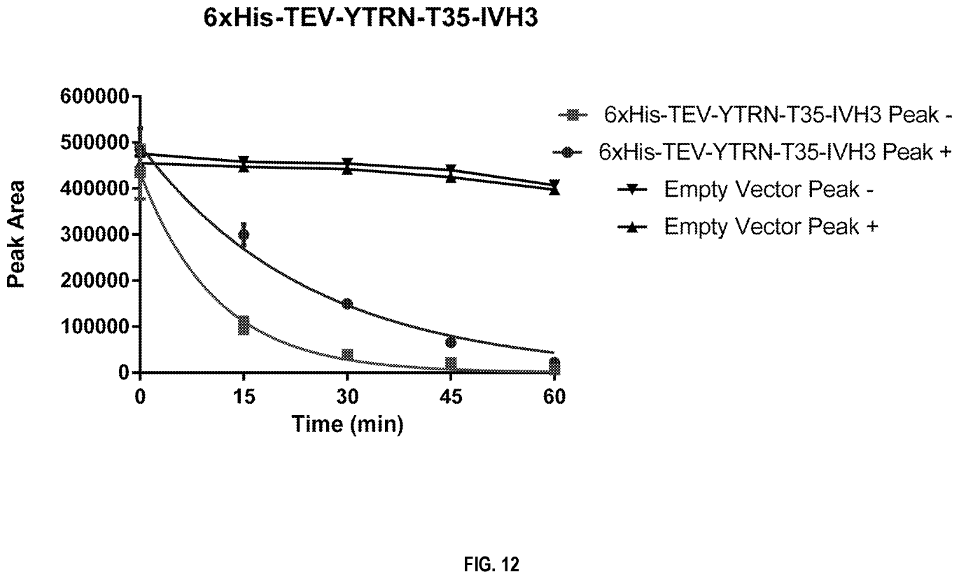

The resulting OPH/IVH3 T35 heterodimer was tested in a UPC2 assay against cyclosarin (O-methyl cyclohexyl phosphonofluoridate), and compared to untethered OPH (YTRN), OPH T35 homodimer and empty vector control (Table 5; FIG. 12).

TABLE 5

| | Apparent Catalytic | | |
|---|---|---|---|
| Enzyme/ | Efficiency | Relative | Stereoselectivity |
| (GF isomer) | (M⁻¹ min⁻¹) | Change | (S_{P}/R_{P}) |
| YTRN (R_{P}) | 3.02E+05 | — | 1.93 |
| YTRN (S_{P}) | 5.82E+05 | — | |
| OPH/IVH3 (R_{P}) | 7.71E+05 | 2.6 | 2.24 |
| OPH/IVH3 (S_{P}) | 1.73E+06 | 3.0 | |
| OPH T35 (R_{P}) | 5.48E+05 | 1.8 | 2.95 |
| OPH T35 (S_{P}) | 1.62E+06 | 2.8 | |

* S_{P} denotes P(−) stereoisomer; R_{P} denotes P(+) stereoisomer

Embodiments of the invention have been described to explain the nature of the invention. Those skilled in the art may make changes in the details, materials, steps and arrangement of the described embodiments within the principle and scope of the invention, as expressed in the appended claims.

### REFERENCES

1. Lenz, D. E. et al., Chem-Bio Interactions (2005); 157-158:205-210.

2. Kapust, R. B. and Waugh, D.S., Protein Science (1999); 8:1668-1674.

3. Cheng, Y-S. E. et al., PNAS (1990); 87:9660-9664.

4. Gadd, M. S. et al., JBC (2011); 286:42971-42980.

5. Fremont, D. H. et al., Science (1996); 272:1001-1004.

What is claimed is:

1. A phosphotriesterase dimer enzyme comprised of two phosphotriesterase subunits tethered to one another via a polypeptide linker, wherein the polypeptide linker is a polypeptide comprising from about 10 to about 35 amino acids in length.

2. The phosphotriesterase dimer enzyme of claim 1, wherein the polypeptide linker is a polyglycine linker.

3. The phosphotriesterase dimer enzyme of claim 1, wherein the polypeptide linker is from 10 to 35 amino acids in length.

4. The phosphotriesterase dimer enzyme of claim 1, wherein the amino acid linker is either 10 or 35 amino acids in length.

5. The phosphotriesterase dimer enzyme of claim 1, wherein the dimer is a homodimer.

6. The phosphotriesterase dimer enzyme of claim 1, wherein the dimer is a heterodimer.

7. The phosphotriesterase dimer enzyme of claim 1, wherein each of the two phosphotriesterase subunits comprise an amino acid sequence independently selected from the group consisting of: SEQ ID NOS: 1-3, and any variant or functional fragment thereof.

8. The phosphotriesterase dimer enzyme of claim 1, wherein both of the two phosphotriesterase subunits comprise the amino acid sequence of SEQ ID NO: 2 or a variant or functional fragment thereof.

9. The phosphotriesterase dimer enzyme of claim 1, wherein both of the two phosphotriesterase subunits comprise the amino acid sequence of SEQ ID NO: 3 or a variant or functional fragment thereof.

10. The phosphotriesterase dimer enzyme of claim 1, wherein one of the two phosphotriesterase monomers comprises the amino acid sequence of SEQ ID NO: 2 or a variant or functional fragment thereof and another of the two phophotriesterase monomers comprises the amino acid sequence of SEQ ID NO: 3 or a variant or functional fragment thereof.

11. The phosphotriesterase dimer enzyme of claim 1, wherein the phosphotriesterase dimer enzyme comprises an amino acid sequence selected from the group consisting of SEQ ID NOS: 5-9 or a variant or functional fragment thereof.

12. The phosphotriesterase dimer enzyme of claim 1, further comprising at least one affinity tag.

13. The phosphotriesterase dimer enzyme of claim 12, wherein the at least one affinity tag is a HIS tag, a maltose binding protein (MBP), or both a HIS tag and an MBP.

14. The phosphotriesterase dimer enzyme of claim 12, wherein the at least one affinity tag is an MBP comprising the amino acid sequence of SEQ ID NO: 4.

15. An isolated polynucleotide comprising a nucleotide sequence encoding the phosphotriesterase dimer enzyme of claim 1.

16. A viral vector comprising the isolated polynucleotide of claim 15.

17. The viral vector of claim 16, wherein the viral vector is an adeno-associated viral vector.

18. A method of treating organophosphate exposure associated damage in a subject, comprising administering to the subject the viral vector of claim 16.

19. A kit comprising the phosphotriesterase dimer of claim 1.

20. A method of detoxifying a surface, comprising contacting the surface with the phosphotriesterase dimer of claim 1.


SEQUENCE LISTING (14 sequences)

SEQ ID NO: 1 - protein, 365 aa, Brevundimonas diminuta
MQTRRVVLKSAAAAGTLLGGLAGCASVAGSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 2 - protein, 339 aa, Artificial Sequence
  PTE mutant YTRN
MSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDHIPYSAIGLEDNASASALLGNRSWQTRALLIKALIDQGYMKQILVSNDWTFGFSSYVTNIMDVMDRVNPDGMAFIPLRVIPFLREKGVPQETLAGITVTNPARFLSPTLRASLE

SEQ ID NO: 3 - protein, 333 aa, Artificial Sequence
  PTE mutant IVH3
MGDRINTVRGPITISEAGFTLTHEHICGSSAGFLRAWPEFFGSRAALVEKAVRGLRRARAAGVRTIVDVSTFDAGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASELLGIRSWQTRALLIKALIDQGYMKQILVSNDWLFGFSSYVTNIMDVMDSVNPDGMAFIPLRVIPFLREKGVPQETLAGITVTNPARFLSPTLRAS

SEQ ID NO: 4 - protein, 382 aa, Artificial Sequence
  MBP affinity tag
KIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLG

SEQ ID NO: 5 - protein, 688 aa, Artificial Sequence
  YTRN-T10-YTRN
MSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDHIPYSAIGLEDNASASALLGNRSWQTRALLIKALIDQGYMKQILVSNDWTFGFSSYVTNIMDVMDRVNPDGMAFIPLRVIPFLREKGVPQETLAGITVTNPARFLSPTLRASLEGGGGGGGGGGMSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDHIPYSAIGLEDNASASALLGNRSWQTRALLIKALIDQGYMKQILVSNDWTFGFSSYVTNIMDVMDRVNPDGMAFIPLRVIPFLREKGVPQETLAGITVTNPARFLSPTLRASLE

SEQ ID NO: 6 - protein, 713 aa, Artificial Sequence
  YTRN-T35-YTRN
MSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDHIPYSAIGLEDNASASALLGNRSWQTRALLIKALIDQGYMKQILVSNDWTFGFSSYVTNIMDVMDRVNPDGMAFIPLRVIPFLREKGVPQETLAGITVTNPARFLSPTLRASLEGGGGGGGGGGGGGGGGGGGGGGGGGGGGGGGGGGGMSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDHIPYSAIGLEDNASASALLGNRSWQTRALLIKALIDQGYMKQILVSNDWTFGFSSYVTNIMDVMDRVNPDGMAFIPLRVIPFLREKGVPQETLAGITVTNPARFLSPTLRASLE

SEQ ID NO: 7 - protein, 675 aa, Artificial Sequence
  IVH3-T10-IVH3
MGDRINTVRGPITISEAGFTLTHEHICGSSAGFLRAWPEFFGSRAALVEKAVRGLRRARAAGVRTIVDVSTFDAGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASELLGIRSWQTRALLIKALIDQGYMKQILVSNDWLFGFSSYVTNIMDVMDSVNPDGMAFIPLRVIPFLREKGVPQETLAGITVTNPARFLSPTLRASGGGGGGGGGGGDRINTVRGPITISEAGFTLTHEHICGSSAGFLRAWPEFFGSRAALVEKAVRGLRRARAAGVRTIVDVSTFDAGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASELLGIRSWQTRALLIKALIDQGYMKQILVSNDWLFGFSSYVTNIMDVMDSVNPDGMAFIPLRVIPFLREKGVPQETLAGITVTNPARFLSPTLRAS

SEQ ID NO: 8 - protein, 700 aa, Artificial Sequence
  IVH3-T35_IVH3
MGDRINTVRGPITISEAGFTLTHEHICGSSAGFLRAWPEFFGSRAALVEKAVRGLRRARAAGVRTIVDVSTFDAGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASELLGIRSWQTRALLIKALIDQGYMKQILVSNDWLFGFSSYVTNIMDVMDSVNPDGMAFIPLRVIPFLREKGVPQETLAGITVTNPARFLSPTLRASGGGGGGGGGGGGGGGGGGGGGGGGGGGGGGGGGGGGDRINTVRGPITISEAGFTLTHEHICGSSAGFLRAWPEFFGSRAALVEKAVRGLRRARAAGVRTIVDVSTFDAGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASELLGIRSWQTRALLIKALIDQGYMKQILVSNDWLFGFSSYVTNIMDVMDSVNPDGMAFIPLRVIPFLREKGVPQETLAGITVTNPARFLSPTLRAS

SEQ ID NO: 9 - protein, 707 aa, Artificial Sequence
  IVH3-T35-YTRN
MGDRINTVRGPITISEAGFTLTHEHICGSSAGFLRAWPEFFGSRAALVEKAVRGLRRARAAGVRTIVDVSTFDAGRDVSLLAEVSRAADVHIVAATGLWEDPPLSMRLRSVEELTQFFLREIQYGIEDTGIRAGIIKVATNGKATPFQELVLRAAARASLATGVPVTTHTAASQRDGEQQAAIFESEGLSPSRVCIGHSDDTDDLSYLTALAARGYLIGLDGIPHSAIGLEDNASASELLGIRSWQTRALLIKALIDQGYMKQILVSNDWLFGFSSYVTNIMDVMDSVNPDGMAFIPLRVIPFLREKGVPQETLAGITVTNPARFLSPTLRASGGGGGGGGGGGGGGGGGGGGGGGGGGGGGGGGGGGMSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLRAAARASLATGVPVTTHTAASQRDGEQQAAIFESEGLSPSRVCIGHSDDTDDLSYLTALAARGYLIGLDHIPYSAIGLEDNASASALLGNRSWQTRALLIKALIDQGYMKQILVSNDWTFGFSSYVTNIMDVMDRVNPDGMAFIPLRVIPFLREKGVPQETLAGITVTNPARFLSPTLRASLE

SEQ ID NO: 10 - DNA, 2064 bp, Artificial Sequence
  YTRN-T10-YTRN coding sequence
  misc_feature (156)..(156): n = “t” or “c” or “a” or “g”
  misc_feature (678)..(678): n = “t” or “c”
  misc_feature (687)..(687): n = “t” or “c”
  misc_feature (1203)..(1203): n = “t” or “c” or “a” or “g”
  misc_feature (1725)..(1725): n = “t” or “c”
  misc_feature (1734)..(1734): n = “t” or “c”
atgagcattggcacgggtgatcgcattaacacggtccgcggtccgattaccatctccgaagcaggttttacgctgacgcacgaacacatttgcggcagctctgcgggttttctgcgtgcctggccggaatttttcggcagccgcaaagcactggcngaaaaggctgtgcgtggcctgcgtcgcgcgcgtgcggccggtgtgcgcaccatcgtggatgtttctacgtttgatattggccgtgacgtttcactgctggccgaagtctcgcgtgcagctgatgttcatatcgttgccgcaaccggtctgtggtttgacccgccgctgagtatgcgtctgcgctccgttgaagaactgacccagtttttcctgcgcgaaattcaatatggcatcgaagatacgggcattcgtgcgggtattatcaaagttgcgaccacgggcaaggcgaccccgtttcaggaactggttctgcgtgcagctgcgcgcgcaagcctggctaccggcgtcccggtgaccacccataccgcagcaagtcaacgcgatggtgaacagcaagctgcgatcttcgaatccgaaggcctgtcaccgtcgcgtgtgtgtattggtcacagcgatgacaccgatgacctgtcttatctgaccgcactggcggcacgcggttacctgattggcctggatcanatcccgtantcagcaattggcctggaagacaacgctagcgcgtctgccctgctgggtaatcgttcgtggcagacccgcgcactgctgattaaagctctgatcgatcagggctatatgaagcaaatcctggtcagcaacgactggacctttggtttcagttcctacgtgacgaacattatggatgtcatggaccgtgtgaatccggatggcatggcgtttatcccgctgcgtgtgattccgttcctgcgcgaaaaaggtgttccgcaggaaaccctggctggcattacggtgaccaatccggctcgcttcctgtccccgaccctgcgtgcgtccctcgagggcggcggcggcggcggcggcggcggcggcatgagcattggcacgggtgatcgcattaacacggtccgcggtccgattaccatctccgaagcaggttttacgctgacgcacgaacacatttgcggcagctctgcgggttttctgcgtgcctggccggaatttttcggcagccgcaaagcactggcngaaaaggctgtgcgtggcctgcgtcgcgcgcgtgcggccggtgtgcgcaccatcgtggatgtttctacgtttgatattggccgtgacgtttcactgctggccgaagtctcgcgtgcagctgatgttcatatcgttgccgcaaccggtctgtggtttgacccgccgctgagtatgcgtctgcgctccgttgaagaactgacccagtttttcctgcgcgaaattcaatatggcatcgaagatacgggcattcgtgcgggtattatcaaagttgcgaccacgggcaaggcgaccccgtttcaggaactggttctgcgtgcagctgcgcgcgcaagcctggctaccggcgtcccggtgaccacccataccgcagcaagtcaacgcgatggtgaacagcaagctgcgatcttcgaatccgaaggcctgtcaccgtcgcgtgtgtgtattggtcacagcgatgacaccgatgacctgtcttatctgaccgcactggcggcacgcggttacctgattggcctggatcanatcccgtantcagcaattggcctggaagacaacgctagcgcgtctgccctgctgggtaatcgttcgtggcagacccgcgcactgctgattaaagctctgatcgatcagggctatatgaagcaaatcctggtcagcaacgactggacctttggtttcagttcctacgtgacgaacattatggatgtcatggaccgtgtgaatccggatggcatggcgtttatcccgctgcgtgtgattccgttcctgcgcgaaaaaggtgttccgcaggaaaccctggctggcattacggtgaccaatccggctcgcttcctgtccccgaccctgcgtgcgtccctcgag

SEQ ID NO: 11 - DNA, 2139 bp, Artificial Sequence
  YTRN-T35-YTRN coding sequence
  misc_feature (156)..(156): n = “t” or “c” or “a” or “g”
  misc_feature (678)..(678): n = “t” or “c”
  misc_feature (687)..(687): n = “t” or “c”
  misc_feature (1278)..(1278): n = “t” or “c” or “a” or “g”
  misc_feature (1800)..(1800): n = “t” or “c”
  misc_feature (1809)..(1809): n = “t” or “c”
atgagcattggcacgggtgatcgcattaacacggtccgcggtccgattaccatctccgaagcaggttttacgctgacgcacgaacacatttgcggcagctctgcgggttttctgcgtgcctggccggaatttttcggcagccgcaaagcactggcngaaaaggctgtgcgtggcctgcgtcgcgcgcgtgcggccggtgtgcgcaccatcgtggatgtttctacgtttgatattggccgtgacgtttcactgctggccgaagtctcgcgtgcagctgatgttcatatcgttgccgcaaccggtctgtggtttgacccgccgctgagtatgcgtctgcgctccgttgaagaactgacccagtttttcctgcgcgaaattcaatatggcatcgaagatacgggcattcgtgcgggtattatcaaagttgcgaccacgggcaaggcgaccccgtttcaggaactggttctgcgtgcagctgcgcgcgcaagcctggctaccggcgtcccggtgaccacccataccgcagcaagtcaacgcgatggtgaacagcaagctgcgatcttcgaatccgaaggcctgtcaccgtcgcgtgtgtgtattggtcacagcgatgacaccgatgacctgtcttatctgaccgcactggcggcacgcggttacctgattggcctggatcanatcccgtantcagcaattggcctggaagacaacgctagcgcgtctgccctgctgggtaatcgttcgtggcagacccgcgcactgctgattaaagctctgatcgatcagggctatatgaagcaaatcctggtcagcaacgactggacctttggtttcagttcctacgtgacgaacattatggatgtcatggaccgtgtgaatccggatggcatggcgtttatcccgctgcgtgtgattccgttcctgcgcgaaaaaggtgttccgcaggaaaccctggctggcattacggtgaccaatccggctcgcttcctgtccccgaccctgcgtgcgtccctcgagggcggcggcggcggcggcggcggcggcggcggcggcggcggcggcggcggcggcggcggcggcggcggcggcggcggcggcggcggcggcggcggcggcggcggcatgagcattggcacgggtgatcgcattaacacggtccgcggtccgattaccatctccgaagcaggttttacgctgacgcacgaacacatttgcggcagctctgcgggttttctgcgtgcctggccggaatttttcggcagccgcaaagcactggcngaaaaggctgtgcgtggcctgcgtcgcgcgcgtgcggccggtgtgcgcaccatcgtggatgtttctacgtttgatattggccgtgacgtttcactgctggccgaagtctcgcgtgcagctgatgttcatatcgttgccgcaaccggtctgtggtttgacccgccgctgagtatgcgtctgcgctccgttgaagaactgacccagtttttcctgcgcgaaattcaatatggcatcgaagatacgggcattcgtgcgggtattatcaaagttgcgaccacgggcaaggcgaccccgtttcaggaactggttctgcgtgcagctgcgcgcgcaagcctggctaccggcgtcccggtgaccacccataccgcagcaagtcaacgcgatggtgaacagcaagctgcgatcttcgaatccgaaggcctgtcaccgtcgcgtgtgtgtattggtcacagcgatgacaccgatgacctgtcttatctgaccgcactggcggcacgcggttacctgattggcctggatgcnatcccgtantcagcaattggcctggaagacaacgctagcgcgtctgccctgctgggtaatcgttcgtggcagacccgcgcactgctgattaaagctctgatcgatcagggctatatgaagcaaatcctggtcagcaacgactggacctttggtttcagttcctacgtgacgaacattatggatgtcatggaccgtgtgaatccggatggcatggcgtttatcccgctgcgtgtgattccgttcctgcgcgaaaaaggtgttccgcaggaaaccctggctggcattacggtgaccaatccggctcgcttcctgtccccgaccctgcgtgcgtccctcgag

SEQ ID NO: 12 - DNA, 2034 bp, Artificial Sequence
  IVH3-T10-IVH3 coding sequence
atgggcgatcggatcaataccgtgcgcggtcctatcacaatctctgaagcgggtttcacactgactcacgagcacatctgcggcagctcggcaggattcttgcgtgcttggccggagttcttcggtagccgcgcagccctagtggaaaaggctgtgagaggattgcgccgcgccagagcggctggcgtgcgaacgattgtcgatgtgtcgactttcgatgccggtcgcgatgtcagtttattggccgaggtttcgcgggctgccgacgttcatatcgtggcggcgaccggcttgtgggaggacccgccactttcgatgcggttgaggagtgtagaggaactcacacagttcttcctgcgtgagattcaatatggcatcgaagacaccggaattagggcgggcattatcaaggtcgcgaccaatggcaaggcgaccccctttcaggagttagtgttaagggcggccgcccgggccagcttggccaccggtgttccggtaaccactcacacggcagcaagtcagcgcgatggtgagcagcaggccgccatttttgagtccgaaggcttgagcccctcacgagtttgtattggtcacagcgatgatactgacgatttgagctatctcaccgccctcgctgcgcgcggatacctcatcggtctagacggcatcccgcacagtgcgattggtctagaagataatgcgagtgcatcagagctcctgggcatccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtcccacaggaaacgctggcaggcatcactgtgaccaacccggcgcggttcttgtcaccgaccttgcgggcgtcatgaggaggaggaggtggaggtggtggtggaggaggcgatcggatcaataccgtgcgcggtcctatcacaatctctgaagcgggtttcacactgactcacgagcacatctgcggcagctcggcaggattcttgcgtgcttggccggagttcttcggtagccgcgcagccctagtggaaaaggctgtgagaggattgcgccgcgccagagcggctggcgtgcgaacgattgtcgatgtgtcgactttcgatgccggtcgcgatgtcagtttattggccgaggtttcgcgggctgccgacgttcatatcgtggcggcgaccggcttgtgggaggacccgccactttcgatgcggttgaggagtgtagaggaactcacacagttcttcctgcgtgagattcaatatggcatcgaagacaccggaattagggcgggcattatcaaggtcgcgaccaatggcaaggcgaccccctttcaggagttagtgttaagggcggccgcccgggccagcttggccaccggtgttccggtaaccactcacacggcagcaagtcagcgcgatggtgagcagcaggccgccatttttgagtccgaaggcttgagcccctcacgagtttgtattggtcacagcgatgatactgacgatttgagctatctcaccgccctcgctgcgcgcggatacctcatcggtctagacggcatcccgcacagtgcgattggtctagaagataatgcgagtgcatcagagctcctgggcatccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtcccacaggaaacgctggcaggcatcactgtgaccaacccggcgcggttcttgtcaccgaccttgcgggcgtcatgataa

SEQ ID NO: 13 - DNA, 2109 bp, Artificial Sequence
  IVH3-T35-IVH3 coding sequence
atgggcgatcggatcaataccgtgcgcggtcctatcacaatctctgaagcgggtttcacactgactcacgagcacatctgcggcagctcggcaggattcttgcgtgcttggccggagttcttcggtagccgcgcagccctagtggaaaaggctgtgagaggattgcgccgcgccagagcggctggcgtgcgaacgattgtcgatgtgtcgactttcgatgccggtcgcgatgtcagtttattggccgaggtttcgcgggctgccgacgttcatatcgtggcggcgaccggcttgtgggaggacccgccactttcgatgcggttgaggagtgtagaggaactcacacagttcttcctgcgtgagattcaatatggcatcgaagacaccggaattagggcgggcattatcaaggtcgcgaccaatggcaaggcgaccccctttcaggagttagtgttaagggcggccgcccgggccagcttggccaccggtgttccggtaaccactcacacggcagcaagtcagcgcgatggtgagcagcaggccgccatttttgagtccgaaggcttgagcccctcacgagtttgtattggtcacagcgatgatactgacgatttgagctatctcaccgccctcgctgcgcgcggatacctcatcggtctagacggcatcccgcacagtgcgattggtctagaagataatgcgagtgcatcagagctcctgggcatccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtcccacaggaaacgctggcaggcatcactgtgaccaacccggcgcggttcttgtcaccgaccttgcgggcgtcatgaggaggaggaggtggaggtggtggtggaggaggaggtggaggtggtggtggtggtggaggaggtggtggtggaggaggtggtggtggcggtggaggaggtggtggtggcgatcggatcaataccgtgcgcggtcctatcacaatctctgaagcgggtttcacactgactcacgagcacatctgcggcagctcggcaggattcttgcgtgcttggccggagttcttcggtagccgcgcagccctagtggaaaaggctgtgagaggattgcgccgcgccagagcggctggcgtgcgaacgattgtcgatgtgtcgactttcgatgccggtcgcgatgtcagtttattggccgaggtttcgcgggctgccgacgttcatatcgtggcggcgaccggcttgtgggaggacccgccactttcgatgcggttgaggagtgtagaggaactcacacagttcttcctgcgtgagattcaatatggcatcgaagacaccggaattagggcgggcattatcaaggtcgcgaccaatggcaaggcgaccccctttcaggagttagtgttaagggcggccgcccgggccagcttggccaccggtgttccggtaaccactcacacggcagcaagtcagcgcgatggtgagcagcaggccgccatttttgagtccgaaggcttgagcccctcacgagtttgtattggtcacagcgatgatactgacgatttgagctatctcaccgccctcgctgcgcgcggatacctcatcggtctagacggcatcccgcacagtgcgattggtctagaagataatgcgagtgcatcagagctcctgggcatccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtcccacaggaaacgctggcaggcatcactgtgaccaacccggcgcggttcttgtcaccgaccttgcgggcgtcatgataa

SEQ ID NO: 14 - DNA, 2127 bp, Artificial Sequence
  IVH3-T35_YTRN coding sequence
atgggcgatcggatcaataccgtgcgcggtcctatcacaatctctgaagcgggtttcacactgactcacgagcacatctgcggcagctcggcaggattcttgcgtgcttggccggagttcttcggtagccgcgcagccctagtggaaaaggctgtgagaggattgcgccgcgccagagcggctggcgtgcgaacgattgtcgatgtgtcgactttcgatgccggtcgcgatgtcagtttattggccgaggtttcgcgggctgccgacgttcatatcgtggcggcgaccggcttgtgggaggacccgccactttcgatgcggttgaggagtgtagaggaactcacacagttcttcctgcgtgagattcaatatggcatcgaagacaccggaattagggcgggcattatcaaggtcgcgaccaatggcaaggcgaccccctttcaggagttagtgttaagggcggccgcccgggccagcttggccaccggtgttccggtaaccactcacacggcagcaagtcagcgcgatggtgagcagcaggccgccatttttgagtccgaaggcttgagcccctcacgagtttgtattggtcacagcgatgatactgacgatttgagctatctcaccgccctcgctgcgcgcggatacctcatcggtctagacggcatcccgcacagtgcgattggtctagaagataatgcgagtgcatcagagctcctgggcatccgttcgtggcaaacacgggctctcttgatcaaggcgctcatcgaccaaggctacatgaaacaaatcctcgtttcgaatgactggctgttcgggttttcgagctatgtcaccaacatcatggacgtgatggatagcgtgaaccccgacgggatggccttcattccactgagagtgatcccattcctacgagagaagggcgtcccacaggaaacgctggcaggcatcactgtgaccaacccggcgcggttcttgtcaccgaccttgcgggcgtcatgaggaggaggaggtggaggtggtggtggaggaggaggtggaggtggtggtggtggtggaggaggtggtggtggaggaggtggtggtggcggtggaggaggtggtggtatgagcattggcacgggtgatcgcattaacacggtccgcggtccgattaccatctccgaagcaggttttacgctgacgcacgaacacatttgcggcagctctgcgggttttctgcgtgcctggccggaatttttcggcagccgcaaagcactggcggaaaaggctgtgcgtggcctgcgtcgcgcgcgtgcggccggtgtgcgcaccatcgtggatgtttctacgtttgatattggccgtgacgtttcactgctggccgaagtctcgcgtgcagctgatgttcatatcgttgccgcaaccggtctgtggtttgacccgccgctgagtatgcgtctgcgctccgttgaagaactgacccagtttttcctgcgcgaaattcaatatggcatcgaagatacgggcattcgtgcgggtattatcaaagttgcgaccacgggcaaggcgaccccgtttcaggaactggttctgcgtgcagctgcgcgcgcaagcctggctaccggcgtcccggtgaccacccataccgcagcaagtcaacgcgatggtgaacagcaagctgcgatcttcgaatccgaaggcctgtcaccgtcgcgtgtgtgtattggtcacagcgatgacaccgatgacctgtcttatctgaccgcactggcggcacgcggttacctgattggcctggatcacatcccgtactcagcaattggcctggaagacaacgctagcgcgtctgccctgctgggtaatcgttcgtggcagacccgcgcactgctgattaaagctctgatcgatcagggctatatgaagcaaatcctggtcagcaacgactggacctttggtttcagttcctacgtgacgaacattatggatgtcatggaccgtgtgaatccggatggcatggcgtttatcccgctgcgtgtgattccgttcctgcgcgaaaaaggtgttccgcaggaaaccctggctggcattacggtgaccaatccggctcgcttcctgtccccgaccctgcgtgcgtccctcgagtaa